(19) United States
(12) Patent Application Publication
(10) Pub. No.: US 2009/0176726 A1
(43) Pub. Date: Jul. 9, 2009

(54) METHODS FOR TREATING MITF-RELATED DISORDERS

(76) Inventors: David E. Fisher, Newton, MA (US); Richard Lin, Boston, MA (US)

Correspondence Address: FISH & RICHARDSON PC, P.O. BOX 1022, MINNEAPOLIS, MN, 55440-1022, US

(21) Appl. No.: 12/089,975

(22) PCT Filed: Oct. 10, 2006
(86) PCT No.: PCT/US06/39375

§ 371 Date: Feb. 6, 2009

Related U.S. Application Data
(60) Provisional application No. 60/725,907, filed on Oct. 11, 2005.

(51) Int. Cl. A61K 31/7105; C12Q 1/26; G01N 33/53; A61P 35/00

(52) U.S. Cl. 514/44; 435/25; 435/7.1

(57) ABSTRACT

Methods for treating melanoma and other MITF-related disorders by administering a compound that causes an increase in HIF-1 level or activity (e.g., by increasing the level of HIF-1I in a cell) within cells. Such methods include administration of a compound that is a hydroxylase inhibitor, e.g., a prolyl hydroxylase inhibitor that reduces hydroxylation of HIF-1I thereby causing an increase in HIF-1I in the cell. Such treatment can lead to a decrease in MITF activity or expression.

### BACKGROUND

Microphthalmia-associated transcription factor (MITF), a basic-helix-loop-helix-leucine-zipper (bHLHzip) protein, is required for the proper development of melanocytes, osteoclasts, retinal pigment epithelial cells, mast cells and natural killer cells. MITF is involved in survival pathways during normal development as well as during neoplastic growth of many melanomas. MITF plays a role in osteoclast development, and mutations in MITF can result in osteopetrosis resulting from defective osteoclast development.

### SUMMARY

Methods for treating melanoma and other MITF-related disorders are described. Certain methods of the invention decrease MITF activity or expression via the same pathway that hypoxia (or compounds that mimic certain aspects of hypoxia) decreases MITF activity or expression. The methods can also decrease MITF activity or expression via the same pathway that hydroxylase inhibitors decrease MITF activity or expression. Certain of the methods include treating a patient with a compound that causes an increase in HIF-1 level or activity (e.g., by increasing the level of HIF-1α in a cell) within cells. Such methods include administration of a compound that is a hydroxylase inhibitor, e.g., a prolyl hydroxylase inhibitor that reduces hydroxylation of HIF-1α thereby causing an increase in HIF-1α in the cell. Such treatment can lead to a decrease in MITF activity or expression.

Described herein is a method for treating melanoma comprising administering to a patient a compound that increases the level or activity of HIF-1 (or HIF-1α) in cells. In various aspects of the method: the compound decreases the level or activity of MITF in cells, and the cells are melanoma cells, the compound is an inhibitor of a prolyl hydroxylase (e.g., HIF-1 PH).

Also described herein is a method for decreasing the level the level or activity of MITF in a cell, comprising exposing the cell to a compound that increases the level or activity of HIF-1 in cells. In various aspects of the method: the cells are melanoma cells, the cells are osteoclasts, the cells are mast cells, and the compound is an inhibitor of a prolyl hydroxylase (e.g., HIF-1 PH, EGLN1, EGLN2 and/or EGLN3).

Described herein is a method for treating a bone loss disorder (e.g., osteoporosis) comprising administering to a patient a compound that increases the level of HIF-1α in cells. In various aspects of the method: the compound decreases the level of MITF in cells, the cells are osteoclasts, and the compound is an inhibitor of a prolyl hydroxylase (e.g., HIF-1 PH, EGLN1, EGLN2 and/or EGLN3).

Described herein is a method for treating an allergic reaction comprising administering to a patient a compound that increases the level of HIF-1α in cells. In various aspects of the method: the compound decreases the level of MITF in cells, the cells are mast cells; and the compound is an inhibitor of a prolyl hydroxylase (e.g., HIF-1 PH, EGLN1, EGLN2 and/or EGLN3).

### DETAILED DESCRIPTION

Described below are experiments demonstrating that MITF is downregulated in melanoma cells, osteoclasts and mast cells under hypoxia (low O₂). Similarly, MITF mRNA and protein is downregulated in melanoma cells exposed to CoCl₂, a treatment that mimics certain aspects of hypoxia. Hypoxia is known to lead to reduced hydroxylation of a number of proteins, including HIF-1α that are hydroxylated by one or another of a family of prolyl hydroxylases. Since, hydroxylation of HIF-1α leads to its degradation, hypoxia generally leads to increased levels of HIF-1 (or HIF-1α). Studies described below demonstrate that certain prolyl hydroxylase inhibitors increase the level of HIF-1α in cells and downregulate MITF. Moreover, other studies described below demonstrate that overexpression of HIF-1α leads to downregulation of MITF.

Since MITF is involved in melanoma, reducing expression of MITF can be useful in treating melanoma.

Hypoxia inducible factor 1 (HIF-1), a transcriptional activator, is induced by hypoxia. HIF-1 is a heterodimer composed of an oxygen-regulated subunit (HIF-1α) and a constitutively expressed subunit (HEF-1). Thus, reduced levels of HIF-1α can lead to reduced levels of HIF-1 if there is insufficient HIF-1α to heterodimerize with HIF-1β.

In normoxic cells, HIF-1α is rather rapidly degraded by a mechanism that entails ubiquitination by von Hippel-Lindau tumor suppressor (pVHL). HIF-1α is commonly limiting in cells relative to HIF-1β. Thus, the level of active HIF-1 is largely dependent on the level of HIF-1α in a cell. Thus, it is possible to alter the level of HIF-1 in cell by altering the level of HIF-1α in a cell. HIF-1 plays a role in a number of cellular and developmental processes including: proliferation, angiogenesis, and cell cycle arrest.

Both the half-life and transactivation function of HIF-1α are regulated by changes in the cellular oxygen level. There are several amino acid modifications within HIF-1α that are responsible for regulation of HIF-1α by oxygen. Two of the two amino acids (at Pro-564 and at Pro-402) are within what is often called the oxygen-dependent degradation domain (ODD). These amino acids are hydroxylated by a prolyl hydroxylase called HIF-1 PH. The hydroxylated form of HIF-1α is recognized by the VHL ubiquitin-protein complex for targeting to the proteosome and degradation. The other modified amino acid (Asn-803) is within what is often called the C-terminal activation domain. This asparagine is hydroxylated by FIH-1. Hydroxylation here during normoxia interferes with the interaction between HIF-1α and transcriptional coactiviators. Thus, hydroxylation of certain amino acid leads to reduced activity of HIF-1. In the case of hydroxylation of Pro-564 and at Pro-402 within HIF-1α, HIF-1 activity is reduced because HIF-1α is degraded. In the case of hydroxylation of Asn-803 within HIF-1α, HIF-1 activity is reduced because HIF-1 a cannot effectively interact with transcriptional coactivators that are important for HIF-1 activity. In addition, acetylation of HIF-1α at Lys-532 may reduce HIF-1 activity. Finally, HIF-1α can be conjugated to SUMO-1 and this modification may influence HIF-1 activity.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts the results of experiments demonstrating that MITF is down-regulated under hypoxia in melanoma cells and osteoclast precursor cells.

FIGS. 2A and 2B depict the results of experiments demonstrating that MITF is downregulated in human mast cells under hypoxia and in the presence of CoCl₂.

FIGS. 3A and 3B depict the results of experiments demonstrating that MITF mMNA and protein levels in human melanoma cells are reduced following growth in CoCl₂.

FIGS. 4A and 4B depict the results of experiments demonstrating that compounds that stabilize HIFα cause downregulation of MITF.

FIGS. 5A and 5B depict the results of experiments demonstrating that overexpression of HIFα downregulates MITF

FIG. 6 depicts the results of an experiment demonstrating that silencing of HIFα blocks CoCl₂-mediated MITF downregulation.

FIG. 7 schematically depicts the region surrounding the start site of FoxD1 transcription. The location and sequence of two putative HIF-1 recognition sites and one putative ARNT/AHR recognition site was are shown.

FIG. 8 depicts the amino acid sequence of human HIF-1α.

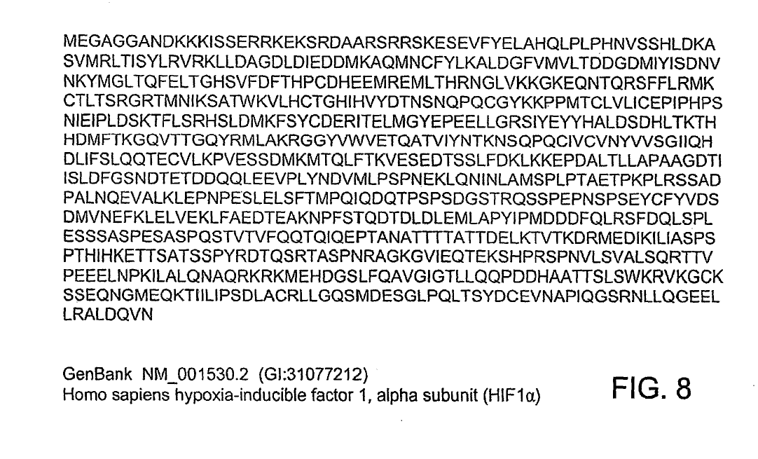

FIG. 9 depicts the amino acid sequence of human Fox-D1

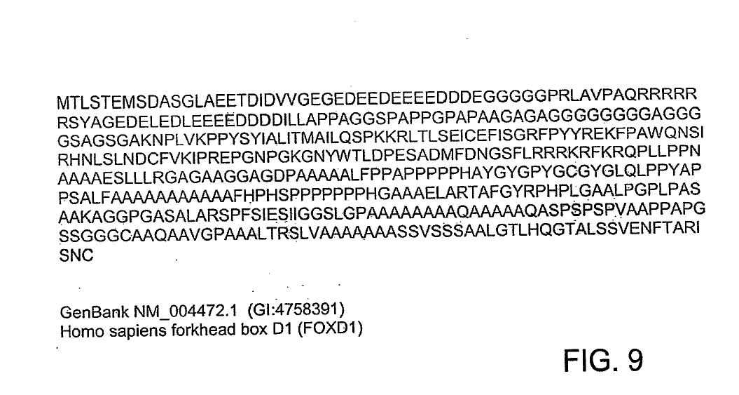

FIG. 10 depicts the amino acid sequence of human HIF-3α

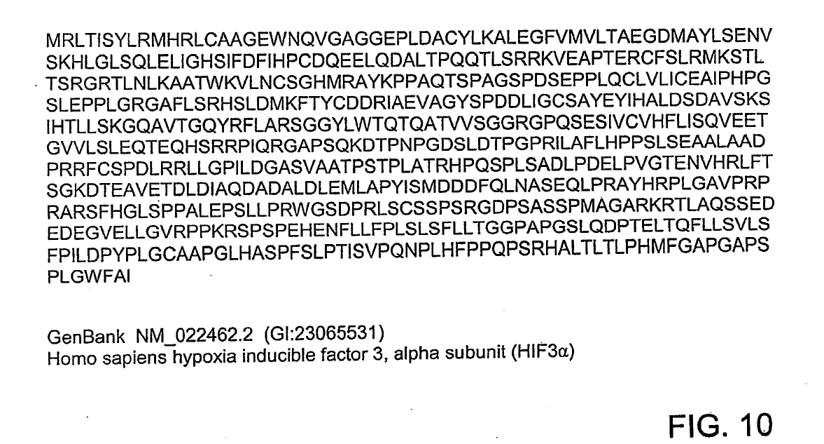

### DETAILED DESCRIPTION

### Example 1

### MITF is Downregulated Under Hypoxia in Melanoma Cells and Osteoclast Precursor Cells

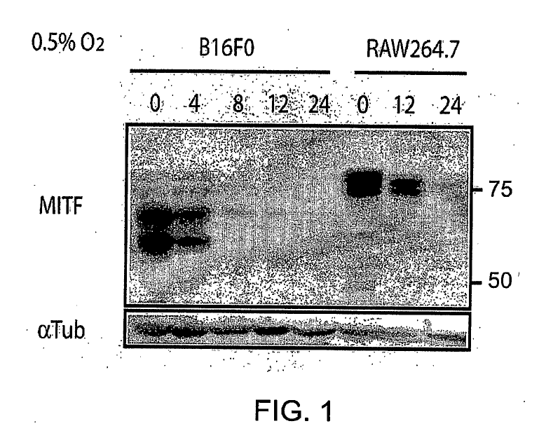

Murine melanoma cells (B16F0 cells) and mouse osteoclast precursor cells (RAW264.7 cells) were grown in DMEM (Mediatech, Inc.) supplemented with 10% fetal bovine serum (FBS) (Sigma) and 1% Penicillin-Streptomycin-Glutamine (PSQ) (Invitrogen, Inc.) under hypoxic conditions (0.5% O₂) MITF protein levels were assessed at 0, 4, 12 and 24 hours using an anti-MITF antibody. As a control α-tubulin protein levels were assessed using an anti-α-tubulin antibody (Sigma). As can be seen in FIG. 1, MITF protein levels steadily decreased under hypoxic conditions in both melanoma cells and osteoclast precursors. The level of α-tubulin was unaffected.

### Example 2

### MITF is Downregulated Under Hypoxia in Human Mast Cells

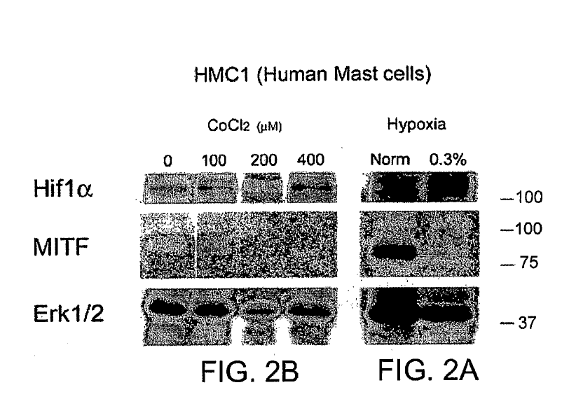

Human mast cells (HMC1 cells) were grown in RPMI-1640 (Mediatech, Inc.) supplemented with 10% fetal bovine serum (FBS) (Sigma) and 1% Penicillin-Streptomycin-Glutamine (PSQ) (Invitrogen), under hypoxic conditions (0.3% O₂). MITF protein levels were assessed at 24 hours using an anti-MITF antibody. As a control Erk1/2 protein levels were assessed using an anti Erk1/2 antibody (Cell Signaling). As can be seen in FIG. 2A, the level of MITF protein was considerably lower in cells grown under hypoxic conditions than in human mast cells grown under normoxic conditions. The level of Erk1/2 was unaffected.

In a separate experiment, human mast cells were grown in RPMI-1640 (Mediatech) supplemented with 10% fetal bovine serum (FBS) (Sigma) and 1% Penicillin-Streptomycin-Glutamine (PSQ) (Invitrogen) without added CoCl₂ or in the presence of 100, 200 or 400 μM CoCl₂ (Sigma). In this experiment MITF protein levels were assessed as above. In addition, HIFα levels were assessed using an anti-HIF-1: antibody (Santa Cruz). CoCl₂ mimics certain aspects of hypoxia, through inhibition of prolyl hydroxylases and stabilization of HIF-1α(Goldberg et al., Science 242:1412-5 (1988); Jaakkola et al., Science 292:468-72 (2001); Yuan et al., J Biol. Chem. 278:15911-6 (2003)), and as can be seen in FIG. 2B, the level of MITF protein decreased as CoCl₂ was increased. As expected, the level of HIF-1α increased as CoCl₂ was increased.

### Example 3

### MITF mRNA and Protein Levels in Human Melanoma Cells are Reduced Following Growth in CoCl₂

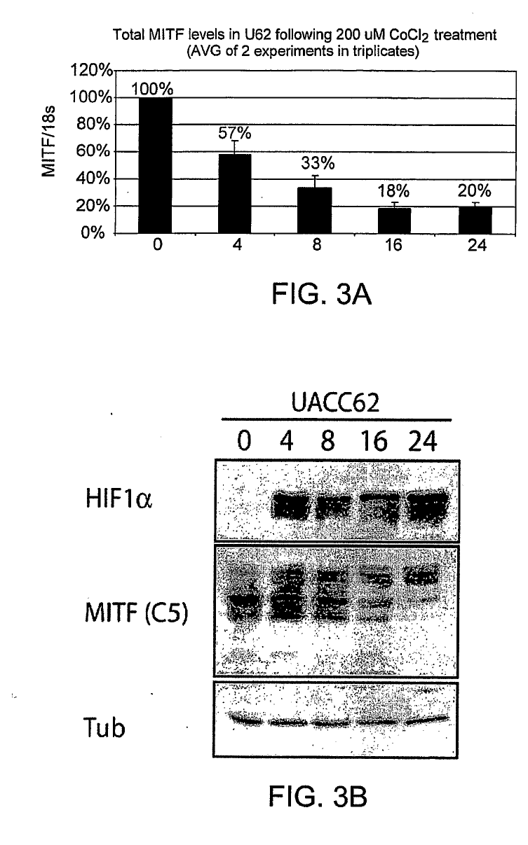

Human melanoma cells (UACC62 cells) were grown in RPMI-1640 Mediatech, Inc.) supplemented with 10% fetal bovine serum (FBS) (Sigma) and 1% Penicillin-Streptomycin-Glutamine (PSQ) (Invitrogen) in the presence of 200 μM CoCl₂. MWrF mRNA was measured at 0, 4, 8, 16 and 24 hrs after addition of CoCl₂ using real time quantitative PCR. As can be seem in FIG. 3A, MITF mRNA levels were decreased following CoCl₂ treatment. MITF and HIF-1α protein levels were also measured using appropriate antibodies. As can seen in FIG. 3B, the MITF protein levels were decreased following CoCl₂ treatment while HIF-1α levels were increased. In this experiment the level of α-tubulin was measured as a control and did not change significantly.

### Example 4

### Compounds that Stabilize HIFα Cause Downregulation of MITF

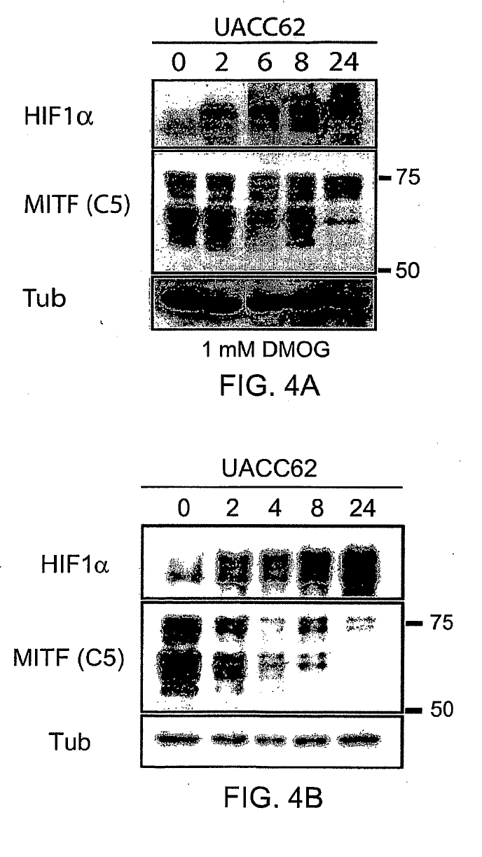

Human melanoma cells (UACC62 cells) were grown in the presence of 1 mM dimethyl-oxalylglycine (DMOG), a prolyl hydroxylase inhibitor (Epstein et al., Cell 107:43-54 (2001); Bruick and McKnight, Science 294:1337-40 (2001)). MITF and HIF-1α protein levels were assessed at 0, 2, 6, 8 and 24 hrs after exposure to DMOG. As can be seen in FIG. 4A, HIF-1α increased steadily from 2 to 8 hours after exposure to DMOG. At 24 hours HIF-1α levels were somewhat lower than at 8 hours, presumably because prolonged DMOG treatment stimulates negative feedback that degrades HIF-1α (Asikainen et al., Proc Natl Acad Sci USA 102:10212-7 (2005)). As can also be seen in FIG. 4A, the level of MITF protein decreased in the presence of DMOG. The level of α-tubulin was measured as a control in this experiment and did not change significantly.

In a separate experiment, human melanoma cells (UACC62 cells) were grown in the presence of 200 μM desferrioxamine (DFO), a compound that chelates iron, a cofactor necessary for prolyl hydroylase activity (Wang and Semenza, Blood 82:3610-5 (1993); Ivan et al., Science 292:464-8 (2001)). MITF and HIF-1α a protein levels were assessed at 0, 2, 6, 8 and 24 hrs after exposure to DFO. As can be seen in FIG. 4B, HIF-1α protein levels increased steadily after exposure to DMOG while MITF protein levels decreased steadily. The level of α-tubulin was measured as a control in this experiment and did not change significantly.

### Example 5

### Overexpression of HIFα Downregulates MITF

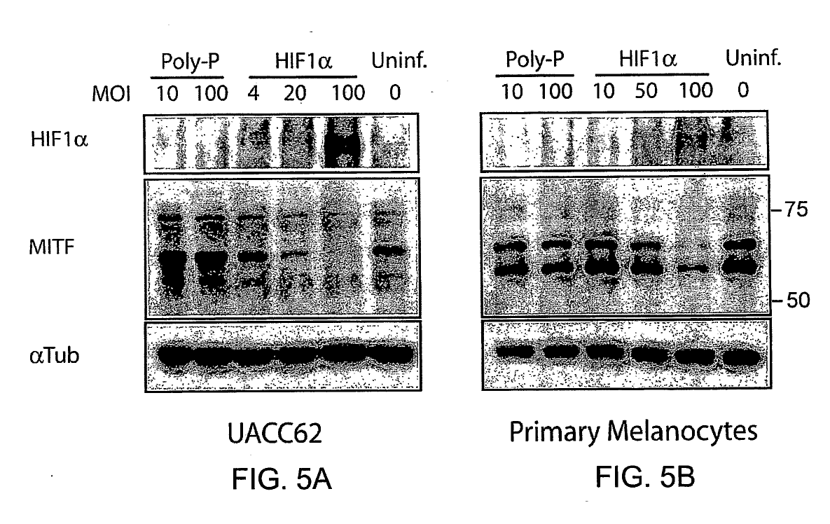

An adenoviral vector was used to overexpress HIF-1α in human melanoma cells (UACC62) and in primary melanocytes. Cells were also infected with an empty virus as a control. MITF protein levels and HIF-1α protein levels were assessed as described above. As can be seen in FIG. 5A, overexpression of HIF-1α caused as decrease in MITF expression in human melanoma cells. The same result was observed in primary melanocytes (see FIG. 5B).

### Example 6

### Silencing of HIFα blocks CoCl₂-Mediated MITF Downregulation

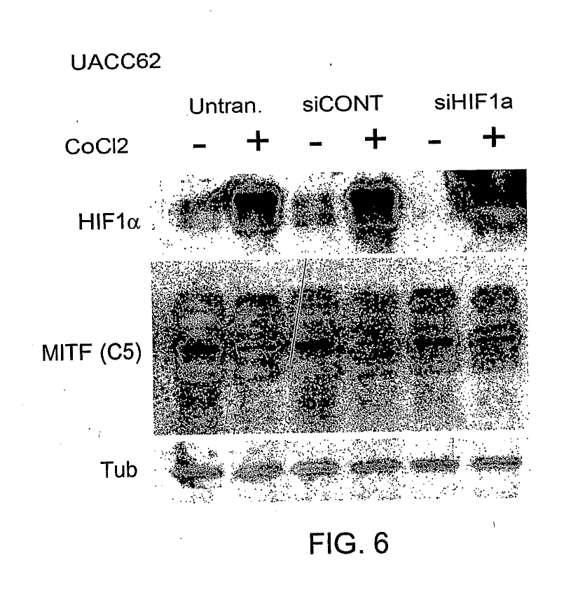

Human melanoma cells (UACC62) were exposed to CoCl₂ in the presence and absence of an siRNA molecule designed to reduce expression of HIF-1α. MITF protein levels and HIF-1α protein levels were measured as described above. As can be seen in FIG. 6, in the absence of a HIFα siRNA, CoCl₂ increased the level of HIF-1α protein and decreased the level of MITF protein. The same result was observed in the presence of a non-specific control siRNA. However, in the presence of an siRNA directed to HIF-1α, downregulation of MITF by CoCl₂ was substantially blocked.

### Example 7

### FoxD1 is Uregulated in Cells Overexpressing HIF-1α

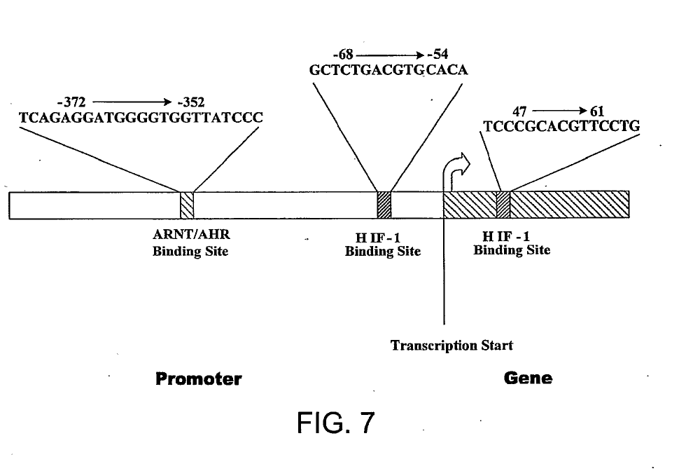

Microarray analysis was performed on human melanoma cells (UACC62) cells exposed to CoCl₂ (induces hypoxia) and on human melanoma cells (UACC62) infected with an adenovirus expressing HIF-1α. In both cases this analysis revealed that FoxD1 is upregulated. FoxD1 (GeneID: 2297; GenBank® Accession No. NM_{—}004472.1; gi:4758391) is a transcriptional regulator that is a member of a family that includes both transcriptional activators and repressors. Moreover, Fox family members are believed to be involved in early neural crest development, when melanocyte differentiation must be suppressed (possibly by suppression of Mitf expression) in order to permit eventual formation of several related cell types (sympathetic neurons, glia, and melanocytes). The region surrounding the FoxD1 transcriptional start site was examined and found to contain two putative HIF-1 recognition sites. The putative recognition sites and their location relative to the HIF-1 transcriptional start site are shown in FIG. 7. A putative ARNT/AHR recognition sequence was also identified and is shown FIG. 7. Based on these results, it is possible that HIF-1 transcriptionally activates expression of FoxD1 which in turn transcriptionally represses expression of MITF. Thus, agents that activate expression of FoxD1 might be useful in the treatment of disorders such as melanoma (or other conditions) where it is desirable to decrease expression of MITF.

### Example 8

### Constitutive Expression of MITF Protects Cells from Cell Death

Human melanoma cells (UACC62) were transfected with a vector that constitutively expresses MITF off a promoter different from its natural promoter. As a control, other cells were transfected with empty vector. Both cell types were exposed to increasing levels of DMOG, which mimics certain aspects of hypoxia in upregulating HIF. This study revealed that cells transformed with a vector that constitutively expresses MITF did not exhibit a detectable decrease in MITF expression when exposed to increasing levels of DMOG. Cells transformed with the empty vector, which still harbor the native MITF gene, exhibited a significant decrease in MITF expression as DMOG levels were increased. Moreover, the cells that constitutively express MITF were protected from cell death at levels of DMOG that were lethal to the cells transformed with empty vector, demonstrating that DMOG's lethal effect in the control cells is due to suppression of MITF.

Assays for Identifying Candidates Compounds for Downregulating MITF

Factors that decrease the expression or activity of a prolyl hydroxylase (e.g., an EGLN) reduce hydroxylation of HIF-1α and thereby lead to increased levels of HIF-1α and this increase in HIF-1α generally leads to a increase in active HIF-1 levels. If increases in HIF-1α directly or indirectly cause a decrease in MITF, candidate compound for downregulating MITF can be identified by screening for inhibition of a prolyl hydroxylase or stabilization of HIF-1α (or HIE-1). The candidate compounds can be optionally tested for their ability to reduce the level of MITF in a cell.

Assays for Identifying Prolyl Hydroxylase Inhibitors

Compounds that inhibit the activity of prolyl hydroxylase, e.g., HIF-1 PH or EGLN2, can be identified using the assays described below. The assays can employ a non-peptide substrate, fully or partially purified polypeptide substrates (purified from cells that naturally express them or produced recombinantly), cells expressing a polypeptide substrate or and/or cell extracts containing a polypeptide substrate. The assays can be used both to identify compounds that decrease hydroxylation of a prolyl hydroxylase substrate, e.g., HIF-1α, and compounds that increase hydroxylation of a prolyl hydroxylase substrate. Where the prolyl hydroxylase is EGLN2, the substrate for the assay can be a human HIF-1α, a natural substrate of EGLN2 hydroxylation, a surrogate EGLN2 substrate or a fragment thereof that is subject to hydroxylation by EGLN2, for example, a human HIF-1α fragment. EGLN2 is expected to catalyze the following reaction, in which R is, for example, HIF-1α and ROH is, for example, hydroxylated HIF-1α.

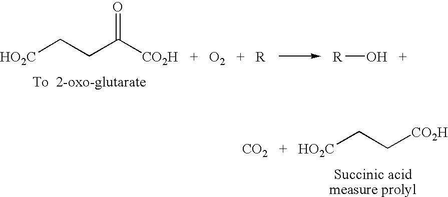

hydroxylase activity the prolyl hydroxylase (e.g., EGLN2) and the substrate of the hydroxylase (e.g., HIF-1α) are contacted in the presence of a co-substrate, such as 2-oxoglutarate (2OG). The hydroxylase activity can be determined, for example, by determining the turnover of the co-substrate. This may be achieved by determining the presence and/or amount of reaction products, such as hydroxylated substrate or succinic acid. The amount of product may be determined relative to the amount of substrate. Thus, hydroxylase activity may be determined by determining the turnover of 2OG to succinate and CO₂ as described in Myllyharju et al. (EMBO J. 16:1173-1180 (1991)) or as in Cunliffe et al (Biochem. J. 240:617-619 (1986)), or other suitable assays for CO₂, bicarbonate or succinate production. To identify an inhibitor of prolyl hydroxylase the assay can be conducted in the presence and absence of a test compound, e.g., a candidate prolyl hydroxylase inhibitor.

A compound which modulates the interaction of HIF-1α or some other substrate of EGLN2 with EGLN2 can be identified by a method comprising: (a) contacting EGLN2 and a test compound in the presence of substrate (e.g., full-length HIF-1α or a fragment thereofthat is subject to hydroxylation) under conditions in which EGLN2 acts on the substrate in the absence of the test compound; and (b) determining the interaction, or lack of interaction, of EGLN2 and the substrate. The interaction of the hydroxylase with the substrate may be determined by measuring the hydroxylation of the substrate (e.g., using a specific antibody or mass spectroscopy) or the binding of the hydroxylase to the substrate or the level of the substrate in a cell. For example, hydroxylation can increase the level of the substrate, e.g., HIF-1α in the cell. The interaction can also be measured by measuring any activity related to the action of the hydroxylase on the substrate, such as the level of a co-factor or by-product used or produced in the hydroxylation reaction, or downstream effects mediated through hydroxylation of the substrate.

The assay can be based on conversion of the substrate into a detectable product. For example, reverse phase HPLC may be used to separate starting synthetic peptide substrates from the hydroxylated products. Thus, the assay can employ mass spectrometric, spectroscopic, and/or fluorescence techniques as are well known in the art (Masimirembwa et al. (2001) Conibinatorial Chemistry & High Throughput Screening 4:245-263, Owicki (2000) J. Biomol. Screen. 5:297-305, Gerslikovich et al. (1996) J. Biochem. Biophys. Meth. 33:135-162, Kraaft et al. (1994) Meth. Eizyiol. 241:70-86). The substrate polypeptide, e.g., HIF-1α or a fragment thereof that is hydroxylated by EGLN2, may be immobilized, e.g., on a bead or plate, and hydroxylation of the appropriate residue detected using an antibody or other binding molecule which binds to the hydroxylated polypeptide with a different affinity than to the non-hydroxylated polypeptide. For example, the antibody recognizes hydroxylated HIF-1α, but binds poorly, if at all, to non-hydroxylated HIFα. Such antibodies can be generated and screened using standard techniques.

Modulators of HIF-1α hydroxylation can also be identified more indirectly by assessing the effect of a test compound on the stability of HIF-1α or the level of HIF-1α or the level of HIF-1 or the activity HIF-1. Thus, assays can be based on identifying an inhibitor of HIF-1αdestruction. Such assays include: (a) providing a substrate (e.g., HIF-1α or a fragment thereof subject to hydroxylation) that includes a hydroxylation site and providing a hydroxylase under conditions suitable for the hydroxylation of a proline residue in the substrate; (b) providing a test compound, e.g., putative modulator of hydroxylation; and (c) determining whether the substrate has been hydroxylated.

A HIF-1α stabilization assay can be carried out using cells expressing HIF-1α as follows. Cells expressing HIF-1α are seeded into 35 mm culture dishes and grown at 37° C., 20% O₂, 5% CO₂ in standard culture medium, e.g., DMEM, 10% FBS. When cell layers reach confluence, the media is replaced with OPTI-MEM media (Invitrogen Life Technologies, Carlsbad Calif.) and cell layers are incubated for approximately 24 hours at 37° C., 20% O₂, 5% CO₂. A test compound in DMSO or 0.013% DMSO is added to existing medium, and incubation is continued overnight. Following overnight incubation, the media is removed and the cells are washed two times in cold phosphate buffered saline (PBS) and then lysed in 1 ml of 10 mM Tris (pH 7.4), 1 mM EDTA, 150 mM NaCl, 0.5% IGEPAL (Sigma-Aldrich, St. Louis Mo.), and a protease inhibitor mix (Roche Molecular Biochemicals) for 15 minutes on ice. Cell lysates are centrifuged at 3,000×g for 5 minutes at 4° C., and the cytosolic fractions (supernatant) are collected. The nuclei (pellet) is resuspended and lysed in 100 μl of 20 mM HEPES (pH 7.2), 400 mM NaCl, 1 mM EDTA, 1 mM dithiothreitol, and a protease mix (Roche Molecular Biochemicals), centrifuged at 13,000×g for 5 minutes at 4° C., and the nuclear protein fractions (supernatant) are collected and analyzed for HIF-1α using a QUANTIKINE immunoassay (R&D Systems, Inc., Minneapolis Minn.) according to the manufacturer's instructions.

Assays which entail measuring the hydroxylation of a substrate (e.g., HIF-1α or a fragment thereof subject to hydroxylation) are carried out under conditions in which the hydroxylase can catalyze hydroxylation. Suitable conditions may include pH 6.6 to 8.5 in an appropriate buffer (for example, Tris HCl or MOPS) in the presence of 2-oxoglutarate, dioxygen and preferably ascorbate and ferrous iron. Reducing agents such as dithiothreitol or tris(carboxyethyl)phosphine may also be present to optimize activity. Other enzymes such as protein disulphide isomerase may be used for the optimization of activity. The enzymes, such as protein disulphide isomerase, may be added in purified or unpurified form. Further components capable of promoting or facilitating the activity of protein disulphide isomerase may also be added.

The format of any of the screening or assay methods may be varied by those of skill in the art. The assays may involve monitoring for hydroxylation of a suitable substrate (in particular monitoring for prolyl hydroxylation), monitoring for the utilization of substrates and co-substrates, monitoring for the production of the expected products between the enzyme and its substrate. Assay methods may also involve screening for the direct interaction between components in the system. Alternatively, assays may be carried out which monitor for downstream effects such as subsequent destruction of HIF-1α, alterations to the levels of HIF-1α in the system and downstream effects mediated by HIF-1 such as HIF-1 mediated transcription using suitable reporter constructs or by monitoring for the upregulation of genes or alterations in the expression patterns of genes know to be regulated directly or indirectly by HIF-1.

The substrate, enzyme and potential inhibitor compound may be incubated together under conditions which in the absence of inhibitor provide for hydroxylation a proline within a polypeptide substrate and the effect of the inhibitor may be determined by determining hydroxylation of the substrate. This may be accomplished by any suitable means. Small polypeptide substrates may be recovered and subject to physical analysis, such as mass spectrometry or chromatography, or to functional analysis, such as the ability to bind to VHL (or displace a reporter molecule from VHL) and be targeted for destruction.

The binding of a substrate to a hydroxylase, e.g., EGLN2, can be assessed in vitro by labeling one component with a detectable label and bringing it into contact with the other component which has been immobilized on a solid support. Suitable detectable labels include ³⁵S which may be incorporated into recombinantly produced peptides and polypeptides. Recombinantly produced peptides and polypeptides may also be expressed as a fusion protein containing an epitope which can be labeled with an antibody. Fusion proteins can incorporate six histidine residues at either the N-terminus or C-terminus of the recombinant protein. Such a histidine tag may be used for purification of the protein by using commercially available columns which contain a metal ion, either nickel or cobalt. These tags also serve for detecting the protein using commercially available monoclonal antibodies directed against the six histidine residues. The protein which is immobilized on a solid support may be immobilized using an antibody against that protein bound to a solid support or the protein can be immobilized using other standard methods. A preferred in vitro interaction may utilize a fusion protein including glutathione-S-transferase (GST). This may be immobilized on glutathione agarose beads. In an in vitro assay format of the type described above, a test compound can be assayed by determining its ability to diminish the amount of labeled peptide or polypeptide which binds to the immobilized GST-fusion polypeptide. This may be determined by fractionating the glutathione-agarose beads by SDS-polyacrylamide gel electrophoresis. Alternatively, the beads may be rinsed to remove unbound protein and the amount of protein which has bound can be determined for example, by counting the amount of label present. The assay can be performed in vivo. The in vivo assay may be performed in a cell line such as a yeast strain in which the relevant polypeptides or peptides are expressed from one or more vectors introduced into the cell.

In some cases it can be useful to measure the binding of VHL to HIF-1α as a measure of hydroxylation, for example, to detect or quantify hydroxylated HIF-1α. The VHL is preferably human VHL (GenBank® Accession Numbers AF010238 and L15409). Other mammalian vHL (e.g., mouse: GenBank Accession number U12570; rat: GenBank Accession numbers U14746 and S80345; or C. elegans VHL (GenBank Accession number F08G12.4) might be useful in some circumstances. It may be possible to use a variant VHL or fragment of VHL that retains the ability to interact directly with a hydroxylated HIF-1α. The ability of VHL fragments and variants to bind to a HIF-1α may be tested as described below.

VHL gene sequences may also be obtained by routine cloning techniques. A wide variety of techniques are available for this, for example, PCR amplification and cloning of the gene using a suitable source of mRNA (e.g., from an embryo or a liver cell), obtaining a cDNA library from a mammalian, vertebrate, invertebrate or fungal source, e.g., a cDNA library from one of the above-mentioned sources, probing the library with a polynucleotide of the invention under stringent conditions, and recovering a cDNA encoding all or part of the VHL protein of that mammal. It is not necessary to use the entire VHL protein in the assay (including their mutants and other variants). Fragments of the VHL may be used, provided such fragments retain the ability to interact with the target domain of the HIF-1α. Generally fragments will be at least 40, preferably at least 50, 60, 70, 80 or 100 amino acids in size. Fragments of HIF-1α may be used, provided that the fragments retain the ability to interact with a wild-type VHL, preferably wild-type human VHL. Such fragments are desirably at least 20, preferably at least 40, 50, 75, 100, 200, 250 or 300 amino acids in size. The fragment retains the proline hydroxylation site. The amount of VHL and HIF-1α may be varied depending upon the scale of the assay. In general, relatively equimolar amounts of the two components are used.

Where assays are performed within cells, the cells may be treated to provide or enhance a normoxic environment, i.e., an oxygen level similar to that found in normal air at sea level. As a control cells may also be cultured under hypoxic conditions, e.g., oxygen levels at 0.1 to 1.0%. The cells may also be treated with compounds which mimic hypoxia and cause up regulation of HIFα. Such compounds include iron chelators (desferrioxamine, O-phenanthroline or hydroxypyridinones (e.g. 1,2-diethyl hydroxypyridinone (CP94) or 1,2-dimethyl hydroxypyridinone (CP20)), cobalt (11), nickel (II) or manganese (II). For cell based assays the proteins may be expressed eukaryotic cells, such as yeast, insect, mammalian, primate and human cells.

Prolyl Hydroxylase Inhibitors

Compounds which may be screened using the assay methods described herein may be natural or synthetic chemical compounds. Extracts of plants, microbes or other organisms, which contain several characterized or uncharacterized components may also be used. Combinatorial libraries (including solid phase synthesis and parallel synthesis methodologies) provide an efficient way of testing larges numbers of different substances for ability to modulate hydroxylation

Small molecule compounds which may be used include 2-oxoglutarate analogues, inhibitors of HIFα such as dimethyl-oxalylglycine, N-oxalylglycine, N-oxalyl-2S-alanine, N-oxalyl-2R-alanine, an enantiomer of N-oxalyl-2S-alanine a potential inhibitors of EgIN3. Other N-oxalyl-amino acid compounds are among the potentially usefual inhibitors.

Warshakoon et al. (Bioorg. Med. Chem. Lett. 16(21):5616-20, 2006; 12 Aug. 2006, e-pub) describe the design and synthesis of substituted pyridine carboxamide derivative (e.g., derivatives having a substituted aryl group at the 5 position of the pyrimidine ring) that are HIF-1α prolyl hydroxylase inhibitors. Warshakoon et al. (Bioorg. Med. Chem. Lett. 16(21):5687-90, 2006; 12 Aug. 2006, e-pub) describe a series of pyrazolopyridines that are potent prolyl hydroxylase inhibitors that are effective in stabilizing HIF-1α. Warshakoon et al. (Bioorg. Med. Chem. Lett. 16(21):5598-601, 2006; 7 Sep. 2006, e-pub) describe a series of imidazo[1,2-a]pyridine derivatives that are EGLN-1 (prolyl hydroxylase) inhibitors. Warshakoon et al. (Bioorg. Med. Chem. Lett. 16(21):5517-22, 2006; 21 Aug. 2006, e-pub) describe 8 hydroxyquinolines that are HIF-1α prolyl hydroxylase inhibitors.

Compounds which stabilize HIFα, apparently by inhibiting a prolyl hydroxylase are described in the following references: Majamaa et al. (Eur. J. Biochem 138:239, 1984); Majamaa et al. (Biochem. J. 229:127, 1985); Bickel et al. (Hepatology 28:4004, 1998); Friedman et al. (Proc. Nat.'l. Acad. Sci. USA 97:4736, 2000); and Franklin et al. (Biochem, J 353:333, 2001).

HIFα stabilizers are described in WO 03/049686; WO 02/074981; WO 03/080566; and WO 04/108681.

Suitable prolyl inhibitors which may be useful to treat MITF-related disorders include those described in US 2004/0254215. For example, suitable inhibitors can have the formula:

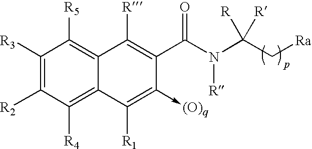

wherein:

q is zero or one;

p is zero or one;

Ra is —COOH or —WR₈; provided that when Rₐ is —COOH then p is zero and when Rₐ is —WR₈ then p is one;

W is selected from the group consisting of oxygen, —S(O)n- and —NR₉— where n is zero, one or two, R₉ is selected from the group consisting of hydrogen, alkyl, substituted alkyl, acyl, aryl, substituted aryl, heteroaryl, substituted heteroaryl, heterocyclic and substituted heterocyclic and R₈ is selected from the group consisting of hydrogen, alkyl, substituted alkyl, aryl, substituted aryl, heteroaryl, substituted heteroaryl, heterocyclic and substituted heterocyclic, or when W is —NR₉— then R₈ and R₉, together with the nitrogen atom to which they are bound, can be joined to form a heterocyclic or a substituted heterocyclic group, provided that when W is —S(O)n- and n is one or two, then R₈ is not hydrogen;

R₁ is selected from the group consisting of hydrogen, alkyl, substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR₆ where X is oxygen, —S(O)n- or —NR₇— where n is zero, one or two, R₆ is selected from the group consisting of alkyl, substituted alkyl, aryl, substituted aryl, heteroaryl, substituted heteroaryl, heterocyclic and substituted heterocyclic, and R₇ is hydrogen, alkyl or aryl or, when X is —NR₇—, then R₇ and R₈, together with the nitrogen atom to which they are bound, can be joined to form a heterocyclic or substituted heterocyclic group;

R₂ and R₃ are independently selected from the group consisting of hydrogen, alkyl, substituted alkyl, aryl, substituted aryl, heteroaryl, substituted heteroaryl, halo, hydroxy, cyano, —S(O)n-(R₆)—R₆ where n is 0, 1, or 2, —NR₆C(O)NR₆, —XR₆ where X is oxygen, —S(O)n- or —NR₇— where n is zero, one or two, each R₆ is independently selected from the group consisting of hydrogen, alkyl, substituted alkyl, aryl, substituted aryl, cycloalkyl, substituted cycloalkyl, heteroaryl, substituted heteroaryl, heterocyclic and substituted heterocyclic provided that when X is —SO— or —SO₂—, then R₆ is not hydrogen, and R₇ is selected from the group consisting of hydrogen, alkyl, aryl, or R₂, R₃ together with the carbon atom pendent thereto, form an aryl substituted aryl, heteroaryl, or substituted heteroaryl;

R₄ and R₅ are independently selected from the group consisting of hydrogen, halo, alkyl, substituted alkyl, alkoxy, substituted alkoxy, aryl, substituted aryl, heteroaryl, substituted heteroaryl and —XR₆ where X is oxygen, —S(O)n- or —NR₇— where n is zero, one or two, R₆ is selected from the group consisting of alkyl, substituted alkyl, aryl, substituted aryl, heteroaryl, substituted heteroaryl, heterocyclic and substituted heterocyclic, and R₇ is hydrogen, alkyl or aryl or, when X is —NR₇—, then R₇ and R₈, together with the nitrogen atom to which they are bound, can be joined to form a heterocyclic or substituted heterocyclic group;

R is selected from the group consisting of hydrogen, deuterium and methyl;

R′ is selected from the group consisting of hydrogen, deuterium, alkyl and substituted alkyl; alternatively, R and R′ and the carbon pendent thereto can be joined to form cycloalkyl, substituted cycloalkyl, heterocyclic or substituted heterocyclic group;

R″ is selected from the group consisting of hydrogen and alkyl or R″ together with R′ and the nitrogen pendent thereto can be joined to form a heterocyclic or substituted heterocyclic group;

R′″ is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O)n-R₁₀ wherein R₁₀ is selected from the group consisting of alkyl, substituted alkyl, cycloalkyl, substituted cycloalkyl, aryl, substituted aryl, heteroaryl and substituted heteroaryl and n is zero, one or two;

and pharmaceutically acceptable salts, esters and prodrugs thereof. US including:

Among the compounds which have been suggested to be hydroxylase inhibitors are:
{[4-Hydroxy-1-(naphthalen-2-yloxy)-isoquinoline-3-carbonyl]-amino}acetic acid;
{[4-Hydroxy-1-(pyridin-3-yloxy)-isoquinoline-3-carbonyl]-amino}acetic acid;
{[4-Hydroxy-1-(4-methoxy-phenoxy)-isoquinoline-3-carbonyl]-amino}acetic acid;
{[4-Hydroxy-1-(3-methoxy-phenoxy)-isoquinoline-3-carbonyl]-amino}acetic acid;
{[1-(3-Fluoro-phenoxy)-4-hydroxy-isoquinoline-3-carbonyl]-amino}acetic acid;
{[1-(4-Fluoro-phenoxy)-4-hydroxy-isoquinoline-3-carbonyl]-amino}-acetic acid;
{[1-(2-Fluoro-phenoxy)-4-hydroxy-isoquinoline-3-carbonyl]-amino}-acetic acid;
{[4-Hydroxy-1-(2-methoxy-phenoxy)-isoquinoline-3-carbonyl]-amino}-acetic acid;
{[1-(4-Acetylamino-phenoxy)-4-hydroxy-isoquinoline-3-carbonyl]-amino}acetic acid;
{[4-Hydroxy-1-(4-methanesulfonylamino-phenoxy)-isoquinoline-3-carbonyl]-amino}acetic acid;
(4-Hydroxy-1-phenylamino-isoquinoline-3-carbonyl)-amino]-acetic acid;
{[4-Hydroxy-6-(pyridin-3-yloxy)-isoquinoline-3-carbonyl]-amino}-acetic acid;
{[4-Hydroxy-7-(pyridin-3-yloxy)-isoquinoline-3-carbonyl]-amino}-acetic acid;
[(1-Chloro-4-methoxy-isoquinoline-3-carbonyl)-amino]-acetic acid;
[(1-Chloro-4-ethoxy-isoquinoline-3-carbonyl)-amino]-acetic acid;
[(4-Hydroxy-1-methoxy-isoquinoline-3-carbonyl)-amino]-acetic acid;
[(1-Ethoxy-4-hydroxy-isoquinoline-3-carbonyl)-amino]-acetic acid;
[(4-Acetoxy-1-phenyl-isoquinoline-3-carbonyl)-amino]-acetic acid;
[(4-Hydroxy-1-phenyl-isoquinoline-3-carbonyl)-amino]-acetic acid;
[(1-Ethoxy-4-phenyl-isoquinoline-3-carbonyl)-amino]-acetic acid;
[(1-Chloro-4-phenyl-isoquinoline-3-carbonyl)-amino]-acetic acid;
[(4-Phenyl-isoquinoline-3-carbonyl)-amino]-acetic acid;
[(4-Hydroxy-1-methyl-isoquinoline-3-carbonyl)-amino]-acetic acid;
[(4-Hydroxy-1-methoxymethyl-isoquinoline-3-carbonyl)-amino]-acetic acid;
[(1-Dimethylcarbamoyl-4-hydroxy-isoquinoline-3-carbonyl)-amino]-acetic acid;
[(4-Hydroxy-1-methyl-6-phenoxy-isoquinoline-3-carbonyl)-amino]-acetic acid;
[(4-Hydroxy-1-methyl-7-phenoxy-isoquinoline-3-carbonyl)-amino]-acetic acid;
[(4-Benzyloxy-1-methyl-7-phenoxy-isoquinoline-3-carbonyl)-amino]acetic acid;
[(4-Ethoxy-1-methyl-7-phenoxy-isoquinoline-3-carbonyl)-amino]acetic acid;
[(1-Dimethylcarbamoyl-4-hydroxy-7-phenoxy-isoquinoline-3-carbonyl)amino]-acetic acid;
[(4-Hydroxy-1-methoxymethyl-7-phenoxy-isoquinoline-3-carbonyl)-amino]-acetic acid;
[(4-Hydroxy-1-p-tolyl-isoquinoline-3-carbonyl)-amino]-acetic acid;
{[7-(4-Fluoro-phenoxy)-4-hydroxy-1-methyl-isoquinoline-3-carbonyl]amino}acetic acid;
{[1-Chloro-4-hydroxy-7-(4-methoxy-phenoxy)-isoquinoline-3-carbonyl]amino}acetic acid;
{[4-Hydroxy-7-(4-methoxy-phenoxy)-isoquinoline-3-carbonyl]-amino}acetic acid;
{[1-Chloro-4-hydroxy-6-(4-methoxy-phenoxy)-isoquinoline-3-carbonyl]-amino}-acetic acid;
{[4-Hydroxy-6-(4-methoxy-phenoxy)-isoquinoline-3-carbonyl]-amino}-acetic acid;
{[1-Chloro-4-hydroxy-7-(4-trifluoromethyl-phenoxy)-isoquinoline-3-carbonyl]-amino}-acetic acid;
{[4-Hydroxy-7-(4-trifluoromethyl-phenoxy)-isoquinoline-3-carbonyl]-amino}-acetic acid;
{[1-Chloro-4-hydroxy-6-(4-trifluoromethyl-phenoxy)-isoquinoline-3-cabonyl]-amino}-acetic acid;
{[4-Hydroxy-6-(4-trifluoromethyl-phenoxy)-isoquinoline-3-carbonyl]-amino}-acetic acid;
{[1-Chloro-7-(4-fluoro-phenoxy)-4-hydroxy-isoquinoline-3-carbonyl]-amino}-acetic acid;
{[7-(4-Fluoro-phenoxy)-4-hydroxy-isoquinoline-3-carbonyl]-amino}-acetic acid;
{[1-Chloro-6-(4-fluoro-phenoxy)-4-hydroxy-isoquinoline-3-carbonyl]-amino}-acetic acid;
{[6-(4-Fluoro-phenoxy)-4-hydroxy-isoquinoline-3-carbonyl]-amino}-acetic acid;
{[4-Hydroxy-7-(pyridin-4-ylsulfanyl)-isoquinoline-3-carbonyl]-amino-}-acetic acid;
{[4-Hydroxy-6-(pyridin-4-ylsulfanyl)-isoquinoline-3-carbonyl]-amino-}-acetic acid;
[(7-Benzeneslfinyl-4-hydroxy-isoquinoline-3-carbonyl)-amino]-acetic acid;
[(7-Benzenesulfonyl-4-hydroxy-isoquinoline-3-carbonyl)-amino]-acetic acid;
[(6-Benzenesulfinyl-4-hydroxy-isoquinoline-3-carbonyl)-amino]-acetic acid;
[(6-Benzenesulfonyl-4-hydroxy-isoquinoline-3-carbonyl)-amino]-acetic acid;
[(6-Amino-4-hydroxy-isoquinoline-3-carbonyl)-amino]-acetic acid;
{[4-Hydroxy-7-(4-methoxy-benzenesulfonylamino)-isoquinoline-3-carbo-nyl]-amino}-acetic acid;
{[4-Hydroxy-7-(3-phenyl-ureido)-isoquinoline-3-carbonyl]-amino}-ace-tic acid;
{[4-Hydroxy-6-(3-phenyl-ureido)-isoquinoline-3-carbonyl]-amino}-ace-tic acid;
[(4-Hydroxy-1-phenylsulfanyl-isoquinoline-3-carbonyl)-amino]-acetic acid;
{[1-(4-Chloro-phenylsulfanyl)-4-hydroxy-isoquinoline-3-carbonyl]-amino}-acetic acid;
[(4-Hydroxy-1-p-tolylsulfanyl-isoquinoline-3-carbonyl)-amino]-acetic acid;
{[4-Hydroxy-1-(pyridin-2-ylsulfanyl)-isoquinoline-3-carbonyl]-amino}-acetic acid;
{[4-Hydroxy-1-(3-methoxy-phenylsulfanyl)-isoquinoline-3-carbonyl]-amino}-acetic acid;
{[4-Hydroxy-1-(2-methoxy-phenylsulfanyl)-isoquinoline-3-carbonyl]-amino}-acetic acid;
{[4-Hydroxy-1-(naphthalen-2-ylsulfanyl)-isoquinoline-3-carbonyl]-amino}-acetic acid;
[(1-Benzenesulfinyl-4-hydroxy-isoquinoline-3-carbonyl)-amino]-acetic acid;
[(1-Benzenesulfonyl-4-hydroxy-isoquinoline-3-carbonyl)-amino]-acetic acid;
{[4-Hydroxy-7-(pyridin-2-ylsulfanyl)-isoquinoline-3-carbonyl]-amino}-acetic acid;
{[4-Hydroxy-6-(pyridin-2-ylsulfanyl)-isoquinoline-3-carbonyl]-amino}-acetic acid;
[(1-Chloro-4-hydroxy-6,7-diphenoxy-isoquinoline-3-carbonyl)-amino]-acetic acid;
[(4-Hydroxy-6,7-diphenoxy-isoquinoline-3-carbonyl)-amino]-acetic acid;
({4-Hydroxy-7-[4-(toluene-4-sulfonylamino)-phenoxy]-isoquinoline-3-carbonyl}-aminoo)-acetic acid;
{[4-Hydroxy-7-(4-nitro-phenoxy)-isoquinoline-3-carbonyl]-amino}-acetic acid;
[(4-Mercapto-7-phenoxy-isoquinoline-3-carbonyl)-amino]-acetic acid;
[(4-Mercapto-7-trifluoromethyl-isoquinoline-3-carbonyl)-amino]-acetic acid;
{[7-(4-Benzenesulfonylamino-phnoxy)-4-hydroxy-isoquinoline-3-carbonyl]-amino}-acetic acid;
{[4-Hydroxy-7-(4-methanesulfonylamino-phenoxy)-isoquinoline-3-carbonyl]amino}-acetic acid;
{[7-(4-Chloro-phenoxy)-4-hydroxy-isoquinoline-3-carbonyl]-amino}-acetic acid;
{[6-(4-Chloro-phenoxy)-4-hydroxy-isoquinoline-3-carbonyl]-amino}-acetic acid;
{[6-(3-Fluoro-5-methoxy-phenoxy)-4-hydroxy-isoquinoline-3-carbonyl]-amino}-acetic acid;
{[7-(3-Fluoro-5-methoxy-phenoxy)-4-hydroxy-isoquinoline-3-carbonyl]-amino}-acetic acid;
{[7-(3,4-Difluoro-phenoxy)-4-hydroxy-isoquinoline-3-carbonyl]-amino-}-acetic acid;
{[6-(3,4-Difluoro-phenoxy)-4-hydroxy-isoquinoline-3-carbonyl]-amino-}-acetic acid;
{[4-Hydroxy-7-(4-trifluoromethoxy-phenoxy)-isoquinoline-3-carbonyl]-amino}-acetic acid;
{[4-Hydroxy-6-(4-trifluoromethoxy-phenoxy)-isoquinoline-3-carbonyl]-amino}-acetic acid;
2-(S)-{[7-(4-Chloro-phenoxy)-4-hydroxy-isoquinoline-3-carbonyl]-amino}-propionic acid;
2-(S)-{[6-(4-Chloro-phenoxy)-4-hydroxy-isoquinoline-3-carbonyl]-amino}-propionic acid;
2-{[7-(3,4-Difluoro-phenoxy)-4-hydroxy-isoquinoline-3-carbonyl]-amino}-propionic acid;
2-(S)-[(4-Hydroxy-7-phenylsulfanyl-isoquinoline-3-carbonyl)-amino]-propionic acid;
2-(R)-[(4-Hydroxy-7-phenylsulfanyl-isoquinoline-3-carbonyl)-amino]-propionic acid;
2-(R)-[(4-Hydroxy-7-phenoxy-isoquinoline-3-carbonyl)-amino]-propionic acid;
2-(S)-{[4-Hydroxy-7-(4-methoxy-phenoxy)-isoquinoline-3-carbonyl]-amino}-propionic acid;
2-(S)-[(7-Benzenesulfonyl-4-hydroxy-isoquinoline-3-carbonyl)-amino]-propionic acid;
(R)-2-[(4-Hydroxy-1-methoxymethyl-7-phenoxy-isoquinoline-3-carbonyl-)-amino]-propionic acid;
(S)-2-[(4-Hydroxy-1-methoxymethyl-7-phenoxy-isoquinoline-3-carbonyl-)-amino]-propionic acid;
(S)-2-[(4-Mercapto-7-phenoxy-isoquinoline-3-carbonyl)-amino]-propionic acid;
(S)-2-{[1-(4-Chloro-phenylsulfanyl)-4-hydroxy-isoquinoline-3-carbonyl]-amino}-propionic acid;
(R)-2-{[1-(4-Chloro-phenylsulfanyl)-4-hydroxy-isoquinoline-3-carbonyl]-amino}-propionic acid;
[(4-Hydroxy-7-phenylsulfa nyl-isoquinoline-3-carbonyl)-amino]-acetic acid;
[(4-Hydroxy-6-phenylsulfanyl-isoquinoline-3-carbonyl)-amino]-acetic acid;
[(1-Chloro-4-hydroxy-7-phenylsulfanyl-isoquinoline-3-carbonyl)-amino]-acetic acid;
[(1-Chloro-4-hydroxy-6-phenylsulfanyl-isoquinoline-3-carbonyl)-amino]-acetic acid;
[(1-Bromo-4-hydroxy-7-phenylsulfanyl-isoquinoline-3-carbonyl)-amino]-acetic acid;
[(1-Bromo-4-hydroxy-6-phenylsulfanyl-isoquinoline-3-carbonyl)-amino-]-acetic acid;
[(4-Hydroxy-7-phenoxy-isoquinoline-3-carbonyl)-amino]-acetic acid;
[(4-Hydroxy-6-phenoxy-isoquinoline-3-carbonyl)-amino]-acetic acid;
[(1-Chloro-4-hydroxy-7-phenoxy-isoquinoline-3-carbonyl)-amino]-acetic acid;
[(1-Chloro-4-hydroxy-6-phenoxy-isoquinoline-3-carbonyl)-amino]-acetic acid;
[(1-Bromo-4-hydroxy-7-phenoxy-isoquinoline-3-carbonyl)-amino]-acetic acid;
[(1-Bromo-4-hydroxy-6-phenoxy-isoquinoline-3-carbonyl)-amino]-acetic acid;
{[7-(2,6-Dimethyl-phenoxy)-4-hydroxy-isoquinoline-3-carbonyl]-amino}-acetic acid;
{[1-Chloro-7-(2,6-dimethyl-phenoxy)-4-hydroxy-isoquinoline-3-carbon-yl]-amino}-acetic acid;
{[1-Bromo-7-(2,6-dimethyl-phenoxy)-4-hydroxy-isoquinoline-3-carbonyl]-amino}-acetic acid;
[(1-Bromo-7-chloro-4-hydroxy-isoquinoline-3-carbonyl)-amino]-acetic acid;
[(1-Bromo-6-chloro-4-hydroxy-isoquinoline-3-carbonyl)-amino]-acetic acid;
[(1-Bromo-4-hydroxy-7-trifluoromethyl-isoquinoline-3-carbonyl)-amino]-acetic acid;
[(1-Bromo-4-hydroxy-6-trifluoromethyl-isoquinoline-3-carbonyl)-amino]-acetic acid;
[(4-Hydroxy-1-phenoxy-isoquinoline-3-carbonyl)-amino]-acetic acid;
[(1,7-dibromo-4-hydroxy-isoquinoline-3-carbonyl)-amino]-acetic acid;
[(7-Bromo-1-chloro-4-hydroxy-isoquinoline-3-carbonyl)-amino]-acetic acid;
[(6-Bromo-4-hydroxy-isoquinoline-3-carbonyl)-amino]-acetic acid;
[(1-Bromo-7-fluoro-4-hydroxy-isoquinoline-3-carbonyl)-amino]-acetic acid;
[(7-Fluoro-4-hydroxy-isoquinoline-3-carbonyl)-amino]-acetic acid;
[(1-Chloro-7-fluoro-4-hydroxy-isoquinoline-3-carbonyl)-amino]-acetic acid;
[(1-Chloro-4-hydroxy-benzo[g]isoquinoline-3-carbonyl)-amino]-acetic acid;
[(1-Bromo-4-hydroxy-isoquinoline-3-carbonyl)-amino]-acetic acid;
[(4-Hydroxy-6-phenyl-isoquinoline-3-carbonyl)-amino]-acetic acid;
[(4-Hydroxy-7-phenyl-isoquinoline-3-carbonyl)-amino]-acetic acid;
[(1-Chloro-4-hydroxy-6-phenyl-isoquinoline-3-carbonyl)-amino]-acetic acid;
[(1-Chloro-4-hydroxy-7-phenyl-isoquinoline-3-carbonyl)-amino]-acetic acid;
[(1-Bromo-4-hydroxy-6-phenyl-isoquinoline-3-carbonyl)-amino]-acetic acid;
[(1-Bromo-4-hydroxy-7-phenyl-isoquinoline-3-carbonyl)-amino]-acetic acid;
[(4-Hydroxy-5-phenyl-isoquinoline-3-carbonyl)-amino]-acetic acid;
[(4-Hydroxy-8-phenyl-isoquinoline-3-carbonyl)-amino]-acetic acid;
[(1-Chloro-4-hydroxy-5-phenyl-isoquinoline-3-carbonyl)-amino]-acetic acid;
[(1-Chloro-4-hydroxy-8-phenyl-isoquinoline-3-carbonyl)-amino]-acetic acid;
[(1-Bromo-4-hydroxy-5-phenyl-isoquinoline-3-carbonyl)-amino]-acetic acid;
[(1-Bromo-4-hydroxy-8-phenyl-isoquinoline-3-carbonyl)-amino]-acetic acid;
[(1-Ethylsulfanyl-4-hydroxy-isoquinoline-3-carbonyl)-amino]-acetic acid;
{[4-Hydroxy-1-(4-methoxy-phenylsulfanyl)-isoquinoline-3-carbonyl]-amino}-acetic acid;
[(1-Chloro-4-hydroxy-7-iodo-isoquinoline-3-carbonyl)-amino]-acetic acid;
[(1-Chloro-4-hydroxy-6-iodo-isoquinoline-3-carbonyl)-amino]-acetic acid;
[(4-Hydroxy-7-iodo-isoquinoline-3-carbonyl)-amino]-acetic acid;
[(1-Bromo-4-hydroxy-7-methyl-isoquinoline-3-carbonyl)-amino]-acetic acid;
[(1-Bromo-7-butoxy-4-hydroxy-isoquinoline-3-carbonyl)-amino]-acetic acid;
[(1-Bromo-6-butoxy-4-hydroxy-isoquinoline-3-carbonyl)-amino]-acetic acid;
[(6-Benzyloxy-1-chloro-4-hydroxy-isoquinoline-3-carbonyl)-methyl-amino]-acetic acid;
[(1-Chloro-4-hydroxy-isoquinoline-3-carbonyl)-methyl-amino]-acetic acid;
[(1-Chloro-4-hydroxy-6-isopropoxy-isoquinoline-3-carbonyl)-methyl-amino]-acetic acid;
[(1-Chloro-4-hydroxy-7-isopropoxy-isoquinoline-3-carbonyl)-methyl-amino]acetic acid;
[Carboxymethyl-(1-chloro-4-hydroxy-isoquinoline-3-carbonyl)-amino]-acetic acid;
[Carboxymethyl-(1-chloro-4-hydroxy-6-isopropoxy-isoquinoline-3-carbonyl)-amino]-acetic acid;
1-Chloro-4-hydroxy-isoquinoline-3-carboxylic acid (2-amino-ethyl)-amide (trifluoro-acetic acid salt);
1-Chloro-4-hydroxy-isoquinoline-3-carboxylic acid (2-methoxy-ethyl)-amide;
1-Chloro-4-hydroxy-isoquinoline-3-carboxylic acid (2-hydroxy-ethyl)-amide;
1-Chloro-4-hydroxy-isoquinoline-3-carboxylic acid (2-dimethylamino-ethyl)-amide;
1-Chloro-4-hydroxy-isoquinoline-3-carboxylic acid (2-acetylamino-ethyl)-amide;
1-Chloro-4-hydroxy-6-isopropoxy-isoquinoline-3-carboxylic acid (2-hydroxy-ethyl)-amide;
1-Chloro-4-hydroxy-6-isopropoxy-isoquinoline-3-carboxylic acid (2-methoxy-ethyl)-amide;
1-Chloro-4-hydroxy-6-isopropoxy-isoquinoline-3-carboxylic acid (2-amino-ethyl)-amide (trifluoro-acetic acid salt);
1-Chloro-4-hydroxy-6-isopropoxy-isoquinoline-3-carboxylic acid (2-dimethylamino-ethyl)-amide;
1-Chloro-4-hydroxy-7-isopropoxy-isoquinoline-3-carboxylic acid (2-amino-ethyl)-amide (trifluoro-acetic acid salt);
1-Chloro-4-hydroxy-7-isopropoxy-isoquinoline-3-carboxylic acid (2-methoxy-ethyl)amide;
1-Chloro-4-hydroxy-7-isopropoxy-isoquinoline-3-carboxylic acid (2-dimethylamino-ethyl)-amide;
1-Chloro-4-hydroxy-7-isopropoxy-isoquinoline-3-carboxylic acid (2-hydroxy-ethyl)amide;
(S)-2-[(6-Benzyloxy-1-chloro-4-hydroxy-isoquinoline-3-carbonyl)-amino]-propionic acid;
(R)-2-[(1-Chloro-4-hydroxy-isoquinoline-3-carbonyl)-amino]-3-hydroxy-y-propionic acid;
(S)-2-[(1-Chloro-4-hydroxy-isoquinoline-3-carbonyl)-amino]-3-hydroxy-y-propionic acid;
(R)-2-[(1-Chloro-4-hydroxy-6-isopropoxy-isoquinoline-3-carbonyl)-amino]-3-hydroxy-propionic acid;
(S)-2-[(1-Chloro-4-hydroxy-6-isopropoxy-isoquinoline-3-carbonyl)-amino]-3-hydroxy-propionic acid;
(R)-2-[(1-Chloro-4-hydroxy-7-isopropoxy-isoquinoline-3-carbonyl)-amino]-3-hydroxy-propionic acid;
(S)-2-[(1-Chloro-4-hydroxy-7-isopropoxy-isoquinoline-3-carbonyl)-amino]-3-hydroxy-propionic acid;
2-[(1-Chloro-4-hydroxy-isoquinoline-3-carbonyl)-amino]-2-methyl-propionic acid;
2-[(1-Chloro-4-hydroxy-6-isopropoxy-isoquinoline-3-carbonyl)-amino]-1-2-methyl-propionic acid;
(R)-2-[(1-Chloro-4-hydroxy-isoquinoline-3-carbonyl)-amino]-3-(1H-imidazol-4-yl)-propionic acid (trifluoro-acetic acid salt);
(S)-2-[(1-Chloro-4-hydroxy-isoquinoline-3-carbonyl)-amino]-3-(H-imidazol-4-yl)-propionic acid (trifluoro-acetic acid salt);
(R)-2-[(1-Chloro-4-hydroxy-isoquinoline-3-carbonyl)-amino]-3-methyl-butyric acid;
(S)-2-[(1-Chloro-4-hydroxy-isoquinoline-3-carbonyl)-amino]-3-methyl-butyric acid;
(R)-2-[(1-Chloro-4-hydroxy-6-isopropoxy-isoquinoline-3-carbonyl)-amino]-3-methyl-butyric acid;
(S)-2-[(1-Chloro-4-hydroxy-6-isopropoxy-isoquinoline-3-carbonyl)-amino]-3-methylbutyric acid;
(R)-2-[(1-Chloro-4-hydroxy-7-isopropoxy-isoquinoline-3-carbonyl)-amino]-3-methyl-butyric acid;
(S)-2-[(1-Chloro-4-hydroxy-7-isopropoxy-isoquinoline-3-carbonyl)-amino]-3-methyl-butyric acid;
(S)-2-[(6-Benzyloxy-1-chloro-4-hydroxy-isoquinoline-3-carbonyl)-amino]-3-methyl-butyric acid;
(R)-2-[(1-Chloro-4-hydroxy-isoquinoline-3-carbonyl)-amino]-3-phenyl-propionic acid;
(S)-2-[(1-Chloro-4-hydroxy-isoquinoline-3-carbonyl)-amino]-3-phenyl-propionic acid;
(R)-2-[(1-Chloro-4-hydroxy-6-isopropoxy-isoquinoline-3-carbonyl)-amino]-3-phenyl-propionic acid;
(S)-2-[(1-Chloro-4-hydroxy-6-isopropoxy-isoquinoline-3-carbonyl)-amino]-3-phenyl-propionic acid;
(R)-2-[(1-Chloro-4-hydroxy-7-isopropoxy-isoquinoline-3-carbonyl)-amino]-3-phenyl-propionic acid;
(S)-2-[(1-Chloro-4-hydroxy-7-isopropoxy-isoquinoline-3-carbonyl)-amino]-3-phenyl-propionic acid;
(R)-2-[(1-Chloro-4-hydroxy-isoquinoline-3-carbonyl)-amino]-3-(4-hydroxy-phenyl)-propionic acid;
(S)-2-[(1-Chloro-4-hydroxy-isoquinoline-3-carbonyl)-amino]-3-(4-hydroxy-phenyl)-propionic acid;
(R)-2-[(1-Chloro-4-hydroxy-6-isopropoxy-isoquinoline-3-carbonyl)-amino]-3-(4-hydroxy-phenyl)-propionic acid;
(S)-2-[(1-Chloro-4-hydroxy-6-isopropoxy-isoquinoline-3-carbonyl)-amino]-3-(4-hydroxy-phenyl)-propionic acid;
(R)-2-[(1-Chloro-4-hydroxy-7-isopropoxy-isoquinoline-3-carbonyl)-amino]-3-(4-hydroxy-phenyl)-propionic acid;
(S)-2-[(1-Chloro-4-hydroxy-7-isopropoxy-isoquinoline-3-carbonyl)-amino]-3-(4-hydroxy-phenyl)-propionic acid;
(R)-2-[(1-Chloro-4-hydroxy-6-isopropoxy-isoquinoline-3-carbonyl)-amino]-pentanoic acid;
(S)-2-[(1-Chloro-4-hydroxy-6-isopropoxy-isoquinoline-3-carbonyl)-amino]-pentanoic acid;
(R)-1-(1-Chloro-4-hydroxy-isoquinoline-3-carbonyl)-pyrrolidine-2-carboxylic acid;
(S)-1-(1-Chloro-4-hydroxy-isoquinoline-3-carbonyl)-pyrrolidine-2-carboxylic acid;
(R)-1-(1-Chloro-4-hydroxy-6-isopropoxy-isoquinoline-3-carbonyl)-pyrrolidine-2-carboxylic acid;
(S)-1-(1-Chloro-4-hydroxy-6-isopropoxy-isoquinoline-3-carbonyl)-pyrrolidine-2-carboxylic acid;
(R)-6-Amino-2-[(1-chloro-4-hydroxy-isoquinoline-3-carbonyl)-amino]-hexanoic acid (trifluoro-acetic acid salt);
(S)-6-Amino-2-[(1-chloro-4-hydroxy-isoquinoline-3-carbonyl)-amino]-hexanoic acid (trifluoro-acetic acid salt);
(R)-6-Amino-2-[(1-chloro-4-hydroxy-6-isopropoxy-isoquinoline-3-carbonyl)-amino]-hexanoic acid; trifluoroacetic acid salt;
(S)-6-Amino-2-[(1-chloro-4-hydroxy-6-isopropoxy-isoquinoline-3-carb-onyl)-amino]-hexanoic acid (trifluoro-acetic acid salt);
(R)-6-Amino-2-[(1-chloro-4-hydroxy-7-isopropoxy-isoquinoline-3-carbonyl)-amino]-hexanoic acid; trifluoroacetic acid salt;
(S)-6-Amino-2-[(1-chloro-4-hydroxy-7-isopropoxy-isoquinoline-3-carbonyl)-amino]-hexanoic acid (trifluoro-acetic acid salt);
(R)-2-[(1-Chloro-4-hydroxy-isoquinoline-3-carbonyl)-amino]-succinic acid;
(S)-2-[(1-Chloro-4-hydroxy-isoquinoline-3-carbonyl)-amino]-succinic acid;
(R)-2-[(1-Chloro-4-hydroxy-6-isopropoxy-isoquinoline-3-carbonyl)-amino]-succinic acid;
(S)-2-[(1-Chloro-4-hydroxy-6-isopropoxy-isoquinoline-3-carbonyl)-amino]-succinic acid;
(R)-2-[(1-Chloro-4-hydroxy-7-isopropoxy-isoquinoline-3-carbonyl)-amino]-succinic acid;
1-[(1-Chloro-4-hydroxy-isoquinoline-3-carbonyl)-amino]-cyclopropane-carboxylic acid;
1-[(1-Chloro-4-hydroxy-6-isopropoxy-isoquinoline-3-carbonyl)-amino]-cyclopropanecarboxylic acid;
(R)-2-[(6-Benzyloxy-1-chloro-4-hydroxy-isoquinoline-3-carbonyl)-amino]-propionic acid;
(S)-2-[(7-Benzyloxy-1-chloro-4-hydroxy-isoquinoline-3-carbonyl)-amino]-propionic acid;
(R)-2-[(7-Benzyloxy-1-chloro-4-hydroxy-isoquinoline-3-carbonyl)-amino]-propionic acid;
(S)-2-[(1-Chloro-4-hydroxy-isoquinoline-3-carbonyl)-amino]-propionic acid;
(R)-2-[(1-Chloro-4-hydroxy-isoquinoline-3-carbonyl)-amino]-propionic acid;
(S)-2-[(6-Isopropoxy-1-chloro-4-hydroxy-isoquinoline-3-carbonyl)-amino]-propionic acid;
(R)-2-[6-Isopropoxy-1-chloro-4-hydroxy-isoquinoline-3-carbonyl)-amino]-propionic acid;
(S)-2-[(7-Isopropoxy-1-chloro-4-hydroxy-isoquinoline-3-carbonyl)-amino-propionic acid;
(R)-2-[(7-Isopropoxy-1-chloro-4-hydroxy-isoquinoline-3-carbonyl)-amino]propionic acid;
1-Chloro-4-hydroxy-6-isopropoxy-isoquinoline-3-carboxylic acid (2-hydroxy-1-hydroxymethyl-ethyl)-amide;
1-Chloro-4-hydroxy-7-isopropoxy-isoquinoline-3-carboxylic acid (2-hydroxy-1-hydroxymethyl-ethyl)-amide;
1-Chloro-4-hydroxy-isoquinoline-3-carboxylic acid (2-hydroxy-1-hydroxymethyl-ethyl)-amide;
{[7-(3,5-Difluoro-phenoxy)-4-hydroxy-isoquinoline-3-carbonyl]-amino}-acetic acid;
{[6-(3,5-Difluoro-phenoxy)-4-hydroxy-isoquinoline-3-carbonyl]-amino-}-acetic acid;
({7-[4-(4-Fluoro-phenoxy)-phenoxy]-4-hydroxy-isoquinoline-3-carbony-1}-amino)-acetic acid;
({6-[4-(4-Fluoro-phenoxy)-phenoxy]-4-hydroxy-isoquinoline-3-carbony-1}-amino)-acetic acid;
{[7-(3-Chloro-4-fluoro-phenoxy)-4-hydroxy-isoquinoline-3-carbonyl]-amino}-acetic acid;
{[6-(3-Chloro-4-fluoro-phenoxy)-4-hydroxy-isoquinoline-3-carbonyl]-amino}-acetic acid;
(S)-2-{[7-(3-Fluoro-5-methoxy-phenoxy)-4-hydroxy-isoquinoline-3-carbonyl]-amino}-propionic acid;
2-(S)-[(7-Cyclohexyloxy-4-hydroxy-isoquinoline-3-carbonyl)-amino]-propionic acid;
2-(S)-{[7-(4-Fluoro-phenoxy)-4-hydroxy-1-methyl-isoquinoline-3-carbonyl]-amino}-propionic acid;
2-(S)-{[7-(4-Fluoro-phenoxy)-4-hydroxy-isoquinoline-3-carbonyl]amino}-propionic acid;
2-(S)-[(4-Hydroxy-1-methyl-7-phenoxy-isoquinoline-3-carbonyl)-amino]-propionic acid;
2-(S)-[(4-Hydroxy-1-methyl-7-phenylsulfanyl-isoquinoline-3-carbonyl)-amino]-propionic acid;
2-(S)-{[4-Hydroxy-7-(4-trifluoromethyl-phenoxy)-isoquinoline-3-carbonyl]-amino}-propionic acid;
{[7-(4-Chloro-phenoxy)-4-hydroxy-1-methyl-isoquinoline-3-carbonyl]-amino}-acetic acid;
{[6-(4-Chloro-phenoxy)-4-hydroxy-1-methyl-isoquinoline-3-carbonyl]-amino}-acetic acid;
{[7-(3,5-Difluoro-phenoxy)-4-hydroxy-1-methyl-isoquinoline-3-carbonyl]-amino}-acetic acid;
{[4-Hydroxy-7-(4-methoxy-phenoxy)-1-methyl-isoquinoline-3-carbonyl]-1-amino}-acetic acid;
{[4-Hydroxy-6-(4-methoxy-phenoxy)-1-methyl-isoquinoline-3-carbonyl]-1-amino}-acetic acid;
[(6-Cyclohexyloxy-4-hydroxy-isoquinoline-3-carbonyl)-amino]-acetic acid;
[(7-Cyclohexyloxy-4-hydroxy-isoquinoline-3-carbonyl)-amino]-acetic acid;
[(7-Cyclohexyloxy-4-hydroxy-1-methyl-isoquinoline-3-carbonyl)-amino-]-acetic acid;
[(7-Cyclohexylsulfanyl-4-hydroxy-isoquinoline-3-carbonyl)-amino]-acetic acid;
[(7-Cyclohexanesulfonyl-4-hydroxy-isoquinoline-3-carbonyl)-amino]-acetic acid;
[(4-Hydroxy-1-isobutyl-isoquinoline-3-carbonyl)-amino]-acetic acid;
[(4-Hydroxy-1-pyridin-2-yl-isoquinoline-3-carbonyl)-amino]-acetic acid;
[(1-Ethyl-4-hydroxy-7-phenoxy-isoquinoline-3-carbonyl)-amino]-acetic acid;
[(1-Dimethylaminomethyl-4-hydroxy-7-phenylsulfanyl-isoquinoline-3-carbonyl)-amino]-acetic acid;
[(4-Hydroxy-1-methyl-7-phenylsulfanyl-isoquinoline-3-carbonyl)-amino]-acetic acid; and
{[4-Hydroxy-1-methyl-7-(4-trifluoromethyl-phenoxy)-isoquinoline-3-carbonyl]-amino}-acetic acid. Pharmaceutically acceptable salts, esters and prodrugs of the aforementioned compounds are also useful.

A very large number of inhibitors of prolyl 4 hydroxylase have been described (see, e.g., U.S. Pat. Nos. 6,200,971; 5,916,898; 5,719,164; 5,726,305 and 6,093,730) and these inhibitors may be useful for treating MITF-related disorders.

US 2006/0199836 describes thienopyridine compounds said to be capable of increasing the stability of activity of HIF.

Additional Means for Reducing HIF-1 Activity or Levels or Activity As discussed above, the level of HIF-1 is generally dependent on the level of HIF-1α in a cell since HIF-1β is commonly present is excess. Since VHL-mediated degradation can limit available HIF-1α, the level of HIF-1 can be increased by reducing VHL activity. This can be accomplished by administering a peptide that binds to VHL and blocks the binding of hydroxylated HIF-1α to VHL. HIF-1 activity can also be increased by blocking the interaction between VHL and KRAB-A, a protein that represses HIF-1 mediated transcriptional activation (Li et al. 2003 EMBO J 22:1875). Further, ARD-1 acetylates HIF-1 at Lys-532 and this modification regulates the interaction of HIF-1α and VHL (Jeong et al. 2002 Cell 111:709). Thus, inhibitors of ARD-1 acetylation can be used to increase the level of HIF-1α in cells thereby decreasing the level of MITF.

SUMO-1 can be conjugated to HIF-1α (Mazure et al. 2004 Biochem Pharmacol 68:971) and this conjugation may reduce the ability of HIF-1 to activated transcription. Thus, compounds that inhibit conjugation of SUMO-1 to HIF-1α may be useful for increasing the activity of HIF-1 thereby decreasing the level of MWIF.

HIF-1α level or activity can be increased by inhibiting prolyl hydroxylation. Using the various inhibitors described above. In addition, limiting available iron (a required co-factor for prolyl hydroxylases) can inhibit prolyl hydroxylases. Available iron can be limited by administering iron chelators such as defferoxamine, ciclopirox olamine or by administering a transition metal (e.g., copper, nickel or cobalt) that competes for iron binding to a prolyl hydroxylase (Martin et al. 2005 Blood 105:4613). RNAi can be used to reduce HIF-1α levels as well (see, for example, Mazure et al. 2004 Biochem Pharmacol 68:971). Prolyl hydroxylation can be decreased by administering peptides that mimic HIF-1α or the relevant prolyl hydroxylase thereby interfering with the interaction between HIF-1α and the prolyl hydroxylase. It may also be possible to reduce prolyl hydroxylation by depleting ascorbate (vitamin C) by use of agents that reduce vitamin C levels or by dietary restriction. It is known OS-9 interacts with HIF-1α and prolyl hydroxylase and might be required for efficient hydroxylation (Baek et al. 2005 Mol Cell 17:503). Thus, RNAi directed against OS-9 might be useful for reducing hydroxylation of HIF-1α.

FIH-1 is an aspargyl hydroxylase that hydroxylates Asn-803 of HIF-1α (Mahon et al. 2001 Genes Dev 15:2675). This hydroxylation is interferes with the ability of HIF-1 to interact with transcriptional co-activators such as p300/CBP. Thus, an agent that reduces the level or activity of FIH-1 could be useful for increasing the activity of HIF-1 and thereby decreasing the activity of MITF. Various inhibitors of prolyl hydroxylases inhibit the activity of FIH-1. In addition compounds such as oxlylglycine and 3, 4 dihydroxybenzoate can inhibit hydroxylation by FIH-1 to a greater extent than they inhibit hydroxylation by prolyl hydroxylases. The structure of FIH-1 is known and the site of interaction with HIF-1α has been identified (Lee et al. 2003 J Biol Chem 278:7558). Accordingly, one can identify peptides, e.g., peptide that resemble a portion of FIH-1, that block the interaction between HIF-1 and FIH-1. In addition, RNAi directed against FIH-1 can be used to reduce the level of FIH-1 thereby increasing the activity of HIF-1 thereby decreasing the level of MITF.

Under normoxia, HIF-1 expression is induced by nitric oxide (NO) donors such as NOC18 or S-nitrosoglutathione (Kasuno et al. 2004 J Biol Chem 279:2550; Palmer et al. 2000 Mol Pharinacol 58:1197). Thus, NO donor can be used to increase the level of HIF-1 thereby decreasing the level of MITF.

Formulation and Administration of Therapeutic Agents

The modulators of hydroxylation and/or MITF expression or activity can be used alone or in combination with other compounds used to treat various disorders, e.g., cancer. Combination therapies are useful in a variety of situations, including where an effective dose of one or more of the agents used in the combination therapy is associated with undesirable toxicity or side effects when not used in combination. This is because a combination therapy can be used to reduce the required dosage or duration of administration of the individual agents.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The modulator, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

The modulator can be in the form of a pharmaceutically acceptable salt. Such salts are prepared from pharmaceutically acceptable non-toxic bases including inorganic bases and organic bases. Examples of salts derived from inorganic bases include aluminum, ammonium, calcium, copper, ferric, ferrous, lithium, magnesium, manganic salts, manganous, potassium, sodium, zinc, and the like. In some embodiments, the salt can be an ammonium, calcium, magnesium, potassium, or sodium salt. Examples of salts derived from pharmaceutically acceptable organic non-toxic bases include salts of primary, secondary, and tertiary amines, benethamine, N,N′-dibenzylethylenediamine, diethylamine, 2-diethylaminoethanol, 2-dimethylaminoethanol, diethanolamine, ethanolamine, ethylenediamine, N-ethylmorpholine, N-ethylpiperidine, epolamine, glucamine, glucosamine, histidine, hydrabamine, isopropylamine, lysine, methylglucamine, meglumine, morpholine, piperazine, piperidine, polyamine resins, procaine, purines, theobromine, triethylamine, trimethylamine, tiipropylamine, and trolamine, tromethamine. Examples of other salts include arecoline, arginine, barium, betaine, bismuth, chloroprocaine, choline, clemizole, deanol, imidazole, and morpholineethanol. In one embodiment are tris salts.

The modulators of the invention can be administered orally, e.g., as a tablet or cachet containing a predetermined amount of the active ingredient, pellet, gel, paste, syrup, bolus, electuary, slurry, capsule; powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The modulators can also be administered by captisol delivery technology, rectal suppository or parenterally.

The compositions may also optionally include other therapeutic ingredients, anticaking agents, preservatives, sweetening agents, colorants, flavors, desiccants, plasticizers, dyes, and the like. The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen. Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents.

The modulators either in their free form or as a salt can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(F-caprolactone) and poly(alkylene oxide) (U.S. 20030068384) to create a sustained release formulation. Such formulations can be used to implants that release a compound of the invention or another agent over a period of a few days, a few weeks or several months depending on the polymer, the particle size of the polymer, and the size of the implant (see, e.g., U.S. Pat. No. 6,620,422).

The modulators can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g. WO 97/11682) via a liposomal formulation (see, e.g., EP 736299, WO 99/59550 and WO 97/13500), via formulations described in WO 03/094886 or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered transdenmally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115)). The agents can be administered using high-velocity transdermal particle injection techniques using the hydrogel particle formulation described in U.S. 20020061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal administration. The agents can be administered in the form a suppository or by other vaginal or rectal means. The agents can be administered in a transmembrane formulation as described in WO 90/07923. The agents can be administered non-invasively via the dehydrated particles described in U.S. Pat. No. 6,485,706. The agent can be administered in an enteric-coated drug formulation as described in WO 02/49621. The agents can be administered intranasaly using the formulation described in U.S. Pat. No. 5,179,079. Formulations suitable for parenteral injection are described in WO 00/62759. The agents can be administered using the casein formulation described in U.S. 20030206939 and WO 00/06108. The agents can be administered using the particulate formulations described in U.S. 20020034536.

The agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications.

A number of embodiments of the invention have been described. Nevertheless, it will be understood that various modifications may be made without departing from the spirit and scope of the invention. Accordingly, other embodiments are within the scope of the following claims.

The references cited herein are incorporated by reference in their entirety.

1. A method for treating melanoma comprising administering to a patient a compound that increases the level or activity of HIF-1 or HIF-1α in at least a subset of cells of the patient.

2. The method of claim 1 wherein the compound decreases the level or activity of MITF in at least a subset of cells of the patient.

3. The method of claim 1 or claim 2 wherein the subset of cells includes melanoma cells.

4. The method of claim 1 or claim 2 wherein the compound is an inhibitor of a prolyl hydroxylase.

5. The method of claim 4 wherein the prolyl hydroxylase is selected from EGLN1, EGLN2 and EGLN3.

6. A method for decreasing the level the level or activity of MITF in a cell, comprising exposing the cell to a compound that increases the level or activity of HIF-1 in the cell.

7. The method of claim 6 wherein the cells is a melanoma cell.

8. The method of claim 6 wherein the cell is a osteoclast.

9. The method of claim 6 wherein the cell is a mast cell.

10. The method of claim 6 wherein the compound is an inhibitor of a prolyl hydroxylase.

11. The method of claim 10 wherein the prolyl hydroxylase is selected from EGLN1, EGLN2 and EGLN3.

12. A method for treating a bone loss disorder comprising administering to a patient a compound that increases the level of HIF-1 or HIF-1α in at least a subset of cells of the patient.

13. The method of claim 12 wherein the compound decreases the level of MITF in cells.

14. The method of claim 12 or claim 13 wherein the cells are osteoclasts.

15. The method of claim 12 or claim 13 wherein the compound is an inhibitor of a prolyl hydroxylase.

16. The method of claim 15 wherein the prolyl hydroxylase is selected from EGLN1, EGLN2 and EGLN3.

17. A method for treating an allergic reaction comprising administering to a patient a compound that increases the level of HIF in at least a subset of cells of the patient.

18. The method of claim 17 wherein the compound decreases the level of MITF in at least a subset of cells of the patient.

19. The method of claim 17 or claim 18 wherein the subset of cells includes mast cells.

20. The method of claim 17 or claim 18 wherein the compound is an inhibitor of a prolyl hydroxylase.

21. The method of claim 40 wherein the prolyl hydroxylase is selected from EGLN1, EGLN2 and EGLN3.

22. A method for identifying a modulator of MITF level, comprising:
(a) measuring the activity of a prolyl hydroxylase in the presence and absence of a candidate modulator under conditions where the prolyl hydroxylase would hydroxylate a polypeptide substrate in the absence of a prolyl hydroxylase modulator; and
(b) identifying the candidate modulator as a modulator of MITF level if the activity of the prolyl hydroxylase differs in the presence and absence of the candidate modulator.

23. A method for identifying a modulator of MITF level, comprising:
(a) measuring the activity of a prolyl hydroxylase in the presence and in the absence of a candidate modulator under conditions where the prolyl hydroxylase would hydroxylate a polypeptide substrate in the absence of a prolyl hydroxylase modulator;
(b) identifying a candidate modulator that alters the activity of the prolyl hydroxylase;
(c) measuring the expression of MITF by cells expressing a prolyl hydroxylase and MITF in the presence and in the absence of a candidate modulator identified in step (b); and
(d) identifying the candidate modulator as a modulator of MITF level if the level of MITF by the cells differs in the presence and in the absence of the candidate modulator.

24. The method of claim 22 or claim 23 wherein the prolyl hydroxylase is selected from EGLN1, EGLN2 and EGLN3.

25. The method of claim 22 or claim 23 wherein the activity of the prolyl hydroxylase is measured by measuring the hydroxylation of the polypeptide substrate.

26. The method of claim 25 wherein the polypeptide substrate is a fragment of HIFα containing a proline.

27. The method of claim 22 or claim 23 wherein the activity of the prolyl hydroxylase is measured in a cell.

28. The method of claim 23 wherein the MITF or the prolyl hydroxylase or both are recombinantly expressed by the cell expressing MITF and the prolyl hydroxylase.

29. The method of claim 22 or claim 23 wherein the activity of the prolyl hydroxylase is measured in vitro.

30. The method of claim 25 wherein the step of measuring hydroxylation of the polypeptide substrate comprises measuring the binding of a VHL polypeptide to the polypeptide substrate.

31. The method of claim 1 or claim 6 wherein the compound inhibits binding of VHL to HIF-1α

32. The method of claim 1 or claim 6 wherein the compound inhibits binding of VHL to KRAB-A.

33. The method of claim 1 or claim 6 wherein the compound inhibits hydroxylation of Pro-402 or Pro-564 of HIF-1α.

34. The method of claim 1 or claim 6 wherein the compound depletes iron.

35. The method of claim 1 or claim 6 wherein the compound competes with iron for binding to HIF-1α prolyl hydroxylase.

36. The method of claim 1 or claim 6 wherein the compound inhibits acetylation of Lys-532 of HIF-1α.

37. The method of claim 1 or claim 6 wherein the compound is an RNAi molecule that interferes with expression of a prolyl hydroxylase that hydroxylates HIF-1α.

38. The method of claim 1 or claim 6 wherein the compound is an RNAi molecule that interferes with expression of an aspargyl hydroxylase that hydroxylates HIF-1α.

39. The method of claim 1 or claim 6 wherein the compound is an RNAi molecule that interferes with expression of FIH-1.

40. The method of claim 1 or claim 6 wherein the compound interferes with the interaction between FIH-1 and VHL.

41. The method of claim 1 or claim 6 wherein the compound is nitric oxide donor that induces HIF-1.

42. The method of claim 1 or claim 6 wherein the compound interferes with the interaction between OS-9 and a prolyl hydroxylase.
